(19) United States
(12) Patent Application Publication
(10) Pub. No.: US 2007/0233091 A1
(43) Pub. Date: Oct. 4, 2007

(54) MULTI-LEVEL SPHERICAL LINKAGE IMPLANT SYSTEM

(76) Inventors: Bill R. Naifeh, Dallas, TX (US); Arnold Oyola, Northborough, MA (US)

Correspondence Address: CARR LLP (IST), 670 FOUNDERS SQUARE, 900 JACKSON STREET, DALLAS, TX, 75202, US

(21) Appl. No.: 11/678,469

(22) Filed: Feb. 23, 2007

Related U.S. Application Data
(60) Provisional application No. 60/775,877, filed on Feb. 23, 2006.
(60) Provisional application No. 60/793,829, filed on Apr. 21, 2006.
(60) Provisional application No. 60/814,753, filed on Jun. 19, 2006.
(60) Provisional application No. 60/775,879, filed on Feb. 23, 2006.
(60) Provisional application No. 60/883,314, filed on Jan. 3, 2007.
(60) Provisional application No. 60/863,284, filed on Oct. 27, 2006.
(60) Provisional application No. 60/826,763, filed on Sep. 25, 2006.
(60) Provisional application No. 60/826,817, filed on Sep. 25, 2006.
(60) Provisional application No. 60/826,807, filed on Sep. 25, 2006.
(60) Provisional application No. 60/825,078, filed on Sep. 8, 2006.
(60) Provisional application No. 60/831,879, filed on Jul. 19, 2006.
(60) Provisional application No. 60/814,753, filed on Jun. 19, 2006.
(60) Provisional application No. 60/814,943, filed on Jun. 19, 2006.
(60) Provisional application No. 60/793,829, filed on Apr. 21, 2006.
(60) Provisional application No. 60/786,898, filed on Mar. 29, 2006.
(60) Provisional application No. 60/775,877, filed on Feb. 23, 2006.

(51) Int. Cl. A61F 2/30

(52) U.S. Cl. 606/61

(57) ABSTRACT

Disclosed in one embodiment, dynamic braces are used in multiple levels to maintain proper vertebral spacing. Such dynamic braces aid in permitting a substantial range of motion in flexion, extension, rotation, anterior-posterior translation and/or other desired types of spinal motion.

### CROSS-REFERENCED TO RELATED APPLICATIONS

The present application is related to and claims priority from the following commonly assigned patent applications: U.S. Provisional Patent Application 60,775,877, entitled “Multi-Level Spherical Linkage Implant System,” filed on Feb. 23, 2006; U.S. Provisional Patent Application 60793829, entitled “Micro Motion Spherical Linkage Implant System,” filed on Apr. 21, 2006; U.S. patent application Ser. No. 11,443,236, entitled “System and Method for Dynamic Skeletal Stabilization,” filed on May 30, 2006; U.S. Provisional Patent Application 60,814,753, entitled “Multi-Level Spherical Linkage Implant System,” filed on Jun. 19, 2006; the disclosures of which are hereby incorporated by reference.

The present application is related to the following commonly assigned patent applications: U.S. patent application Ser. No. 10,914,751, entitled “System and Method for Dynamic Skeletal Stabilization,” filed on Aug. 9, 2004; U.S. Provisional Patent Application 60,637,324, entitled “Three Column Support Dynamic Stabilization System and Method of Use,” filed on Dec. 16, 2004; U.S. Provisional Patent Application 60,656,126, entitled “System and Method for Dynamic Stabilization,” filed on Feb. 24, 2005; U.S. Provisional Patent Application 60,685,705, entitled “Four-Bar Dynamic Stabilization Device,” filed on May 27, 2005; U.S. Provisional Patent Application 60,685,760, entitled “Slidable Post Dynamic Stabilization Device,” filed on May 27, 2005; U.S. Provisional Patent Application 60,693,300, entitled “Spherical Plate Dynamic Stabilization Device,” filed on Jun. 22, 2005; U.S. Provisional Patent Application 60,692,943, entitled “Spherical Motion Dynamic Spinal Stabilization Device,” filed on Jun. 22, 2005; U.S. Provisional Patent Application 60,711,812, entitled “Dynamic Spinal Stabilization Alignment Instrument,” filed on Aug. 26, 2005; U.S. Provisional Patent Application 11,303,138, entitled “Three Column Support Dynamic Stabilization System and Method,” filed on Dec. 16, 2005; U.S. Provisional Patent Application 60,775,879, entitled “Aligning Cross-Connector,” filed on Feb. 23, 2006; U.S. Provisional Patent Application 60,775,877, entitled “Multi-Level Spherical Linkage Implant System,” filed on Feb. 23, 2006; U.S. Provisional Patent Application 60,786,898, entitled “Full Motion Spherical Linkage Implant System,” filed on Mar. 29, 2006; U.S. Provisional Patent Application 60,793,829, entitled “Micro Motion Spherical Linkage Implant System,” filed on Apr. 21, 2006; U.S. patent application Ser. No. 11,443,236, entitled “System and Method for Dynamic Skeletal Stabilization,” filed on May 30, 2006; U.S. Provisional Patent Application 60,814,943, entitled “Aligning Cross-Connector,” filed on Jun. 19, 2006; U.S. Provisional Patent Application 60,814,753, entitled “Multi-Level Spherical Linkage Implant System,” filed on Jun. 19, 2006; U.S. Provisional Patent Application 60,831,879, entitled “Locking Assembly,” filed on Jul. 19, 2006; U.S. patent application Ser. No. 11,467,798, entitled “Alignment Instrument for Dynamic Spinal Stabilization Systems,” filed on Aug. 28, 2006; U.S. Provisional Patent Application 60,825,078, entitled “Offset Adjustable Dynamic Stabilization System,” filed on Sep. 8, 2006; U.S. Provisional Patent Application 60,826,807, entitled “Offset Adjustable Dynamic Stabilization System,” filed on Sep. 25, 2006; U.S. Provisional Patent Application 60,826,817, entitled “Offset Adjustable Dynamic Stabilization System,” filed on Sep. 25, 2006; U.S. Provisional Patent Application 60,826,763, entitled “Alignment Instrument for Dynamic Spinal Stabilization Systems,” filed on Sep. 25, 2006; U.S. Provisional Patent Application 60,863,284, entitled “Alignment Instrument for Dynamic Spinal Stabilization Systems,” filed on Oct. 27, 2006; and U.S. Provisional Patent Application 60,883,314, entitled “Dynamic Linking Member for Spine Stabilization System,” filed on Jan. 3, 2007, the disclosures of which are hereby incorporated by reference.

### FIELD OF THE INVENTION

This disclosure relates to skeletal stabilization and, more particularly, to systems and method for stabilization of human spines and, even more particularly, to dynamic stabilization techniques.

### BACKGROUND

The human spine is a complex structure designed to achieve a myriad of tasks, many of them of a complex kinematic nature. The spinal vertebrae allow the spine to flex in three axes of movement relative to the portion of the spine in motion. These axes include the horizontal (bending either forward/anterior or aft/posterior), roll (bending to either left or right side) and vertical (twisting of the shoulders relative to the pelvis).

In flexing about the horizontal axis, into flexion (bending forward or anterior) and extension (bending backward or posterior), vertebrae of the spine must rotate about the horizontal axis, to various degrees of rotation. The sum of all such movement about the horizontal axis of produces the overall flexion or extension of the spine. For example, the vertebrae that make up the lumbar region of the human spine move through roughly an arc of 15° relative to its adjacent or neighboring vertebrae. Vertebrae of other regions of the human spine (e.g., the thoracic and cervical regions) have different ranges of movement. Thus, if one were to view the posterior edge of a healthy vertebrae, one would observe that the edge moves through an arc of some degree (e.g., of about 15° in flexion and about 5° in extension if in the lumbar region) centered around a center of rotation. During such rotation, the anterior (front) edges of neighboring vertebrae move closer together, while the posterior edges move farther apart, compressing the anterior of the spine. Similarly, during extension, the posterior edges of neighboring vertebrae move closer together, while the anterior edges move farther apart, compressing the posterior of the spine. Also during flexion and extension, the vertebrae move in horizontal relationship to each other, providing up to 2-3 mm of translation.

In a normal spine, the vertebrae also permit right and left lateral bending. Accordingly, right lateral bending indicates the ability of the spine to bend over to the right by compressing the right portions of the spine and reducing the spacing between the right edges of associated vertebrae. Similarly, left lateral bending indicates the ability of the spine to bend over to the left by compressing the left portions of the spine and reducing the spacing between the left edges of associated vertebrae. The side of the spine opposite that portion compressed is expanded, increasing the spacing between the edges of vertebrae comprising that portion of the spine. For example, the vertebrae that make up the lumbar region of the human spine rotate about an axis of roll, moving through roughly an arc of 10° relative to its neighbor vertebrae, throughout right and left lateral bending.

Rotational movement about a vertical axis relative to the portion of the spine moving is also natural in the healthy spine. For example, rotational movement can be described as the clockwise or counter-clockwise twisting rotation of the vertebrae during a golf swing.

The inter-vertebral spacing (between neighboring vertebrae) in a healthy spine is maintained by a compressible and somewhat elastic disc. The disc serves to allow the spine to move about the various axes of rotation and through the various arcs and movements required for normal mobility. The elasticity of the disc maintains spacing between the vertebrae, allowing room or clearance for compression of neighboring vertebrae, during flexion and lateral bending of the spine. In addition, the disc allows relative rotation about the vertical axis of neighboring vertebrae, allowing twisting of the shoulders relative to the hips and pelvis. Clearance between neighboring vertebrae maintained by a healthy disc is also important to allow nerves from the spinal chord to extend out of the spine, between neighboring vertebrae, without being squeezed or impinged by the vertebrae.

In situations (based upon injury or otherwise) where a disc is not functioning properly, the inter-vertebral disc tends to compress, and in doing so pressure is exerted on nerves extending from the spinal cord by this reduced inter-vertebral spacing. Various other types of nerve problems may be experienced in the spine, such as exiting nerve root compression in the neural foramen, passing nerve root compression, and ennervated annulus (where nerves grow into a cracked/compromised annulus, causing pain every time the disc/annulus is compressed), as examples. Many medical procedures have been devised to alleviate such nerve compression and the pain that results from nerve pressure. Many of these procedures revolve around attempts to prevent the vertebrae from moving too close to each other thereby maintaining space for the nerves to exit without being impinged upon by movements of the spine.

In one such procedure, screws are embedded in adjacent vertebrae pedicles and rigid rods or plates are then secured between the screws. In such a situation, the pedicle screws (which are in effect extensions of the vertebrae) then press against the rigid spacer which serves to distract the degenerated disc space, maintaining adequate separation between the neighboring vertebrae, so as to prevent the vertebrae from compressing the nerves. This prevents nerve pressure due to extension of the spine; however, when the patient then tries to bend forward (putting the spine in flexion), the posterior portions of at least two vertebrae are effectively held together and are not allowed to move as a natural healthy spine. Furthermore, the lateral bending or rotational movement between the affected vertebrae is significantly reduced, due to the rigid connection of the spacers and rods. Overall movement of the spine is reduced as more vertebras are distracted by such rigid spacers. This type of system not only limits the patient's movements, but also places additional stress on other portions of the spine (typically, the stress placed on adjacent vertebrae without spacers being the worse), often leading to further complications at a later date.

In other procedures, dynamic stabilization devices are used. Typically, such devices do not allow multiple levels of stabilization of the vertebrae and do not allow for interchangeability of dynamic and fusion type systems for multiple levels.

What is needed is a dynamic system that provides for dynamic stabilization and/or fusion of the spine at multiple levels, while increasing the ease of insertion by allowing for adjustability of components during implantation and accounting for variations in patient anatomy.

### SUMMARY

In response to these and other problems, there is presented certain aspects which may provide methods and spine stabilization systems for maintaining spacing between multiple consecutive vertebrae, while allowing movement of the vertebrae relative to each other in at least two and preferably three axes of rotation.

In one embodiment, dynamic braces are used in multiple levels to maintain proper vertebral spacing. The dynamic braces are designed to allow the vertebrae to which it is attached to move through natural arc, which may travel on an imaginary surface of a sphere or another curved surface. Accordingly, such dynamic braces aid in permitting a substantial range of motion in flexion, extension, rotation, anterior-posterior translation and/or other desired types of spinal motion.

These and other features, and advantages, will be more clearly understood from the following detailed description taken in conjunction with the accompanying drawings. It is important to note the drawings are not intended to represent the only aspect of the invention.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 is a perspective view of one side of a multi-level dynamic stabilization system;

FIG. 2A is a detailed perspective view of one embodiment of a brace which may be used in the dynamic stabilization system of FIG. 1 illustrated in a neutral position;

FIG. 2B is a perspective view of the brace illustrated in FIG. 1 illustrated in a flexed position;

FIG. 2C is a perspective view of the brace illustrated in FIG. 1 illustrated in a lateral bending position;

FIG. 2D is a perspective view of the brace illustrated in FIG. 1 illustrated in a rotational position;

FIG. 3 is a cross sectional view of a component that may be incorporated in the dynamic stabilization system of FIG. 1 and 4;

FIG. 4 is a perspective view of an alternative embodiment of a multi-level dynamic stabilization system;

FIG. 5 is a detailed perspective view of a component which may be used in the dynamic stabilization system of FIG. 1 and 4;

FIG. 6A is a perspective view of a limiter element which may be incorporated into the dynamic stabilization system of FIG. 1 and 3;

FIG. 6B is an enlarged perspective view of an alternative embodiment of a dynamic brace which may be incorporated into the dynamic stabilization system of FIG. 1 and 4;

FIG. 7 is a perspective view of both sides of a multi-level dynamic stabilization system; and

FIG. 8 is a perspective view of the dynamic stabilization system shown in FIG. 7 implanted in multiple consecutive vertebrae;

FIG. 9 is a perspective view of an alternative embodiment illustrating a connection between a pedicle screw and the rods, which may be incorporated into the dynamic stabilization system of FIG. 1 and 4;

FIG. 10 is a top view of a kit for a multi-level dynamic stabilization system.

### DETAILED DESCRIPTION

For the purposes of promoting an understanding of the principles of the present inventions, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

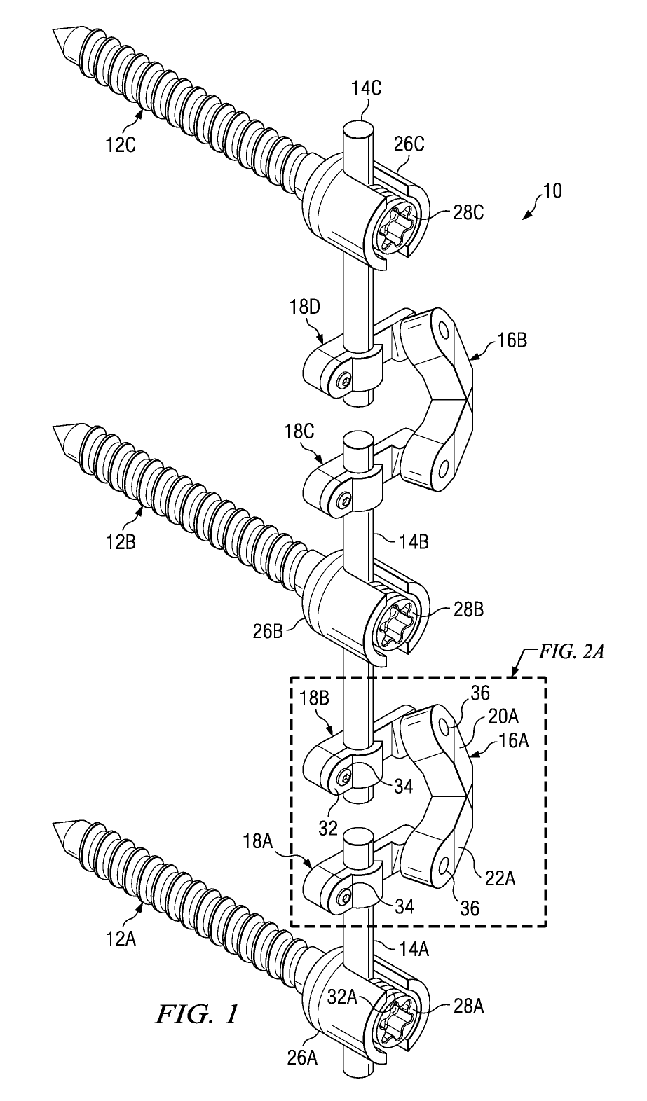

Referring now to FIG. 1, there is shown one embodiment of a spine stabilization system 10 which may be secured to one side of the spine. The spine stabilization system 10 may be used to link and stabilize three or more vertebrae. As illustrated, the spine stabilization system 10 may incorporate three or more bone anchors 12a-12c, three or more anchor heads 26a-26c that couple to the bone anchors 12a-12c and two or more dynamic braces 16a-16b that are positioned between anchor heads 26a-26c via rod or link members. Additionally, the various components of the spine stabilization system 10 may be manufactured from medical grade implantable polymers or metals, such as titanium, PEEK, cobalt chrome, nitinol, and stainless steel. As will be explained below in greater detail, the spine stabilization system 10 provides stabilization for three or more vertebrae, thus enabling multi-level spine stabilization.

Each of the bone anchors 12a-12c may have a distal threaded section that is secured into a patient's vertebrae. In certain embodiments, the proximal end of each bone anchor 12a-12c may be shaped to couple in a polyaxial manner to an anchor head (such as anchor heads 26a-26c). One such example of a bone anchor coupled in a polyaxial manner to a anchor head is disclosed in application Ser. No. 10/990,272 entitled “An Implant Assembly and Method for use in an Internal Structure Stabilization System” filed on Nov. 16, 2004, the disclosure of which is hereby incorporated by reference for all purposes. The bone anchors 12a-12c may be pedicle screws or other suitable bone anchoring devices such as plates, rods, hooks, or nails.

In certain embodiments, the anchor heads 26a-26c may have a generally smooth outer surface and a threaded internal surface. In some embodiments, the anchor heads 26a-26c may have a central hole or bore extending along its longitudinal center axis creating a cylindrical shaped head. The central hole may receive the proximal end of bone anchors 12a-12c from either direction. In certain embodiments the cylindrical shaped head may have an elongated slot on one or both sides of the head which may be perpendicular to the central hole. The elongated slot may be dimensioned to receive one or more rods 14a-14c. In yet other embodiments, which will be described in greater detail below, rods 14a-14c may be shaped so they may couple to anchor heads 26a-26c in a polyaxial manner.

The rods 14a-14c may be adjusted vertically as needed to accommodate various placements of connecting members 18a-18d and, as will be explained later, accommodate a strategic placement of the braces 16a-16b. The rods 14a-14c may also slide within anchor heads 26a-26c to allow for adjustability during implantation. As will be explained below, in certain embodiments the connecting members 18a-18d may also slide relative to the bone anchors 12a-12c to accommodate various distances between bone anchors. The final position of rods 14a-14c and anchor heads 26a-26c may be secured by locking elements 28a-28c. The locking elements 28a-28c may be locking caps or other suitable locking elements known to those skilled in the art. In certain embodiments, the locking elements 28a-28c may have a threaded external surface that mates with a threaded internal surface of the respective anchor heads 26a-26c.

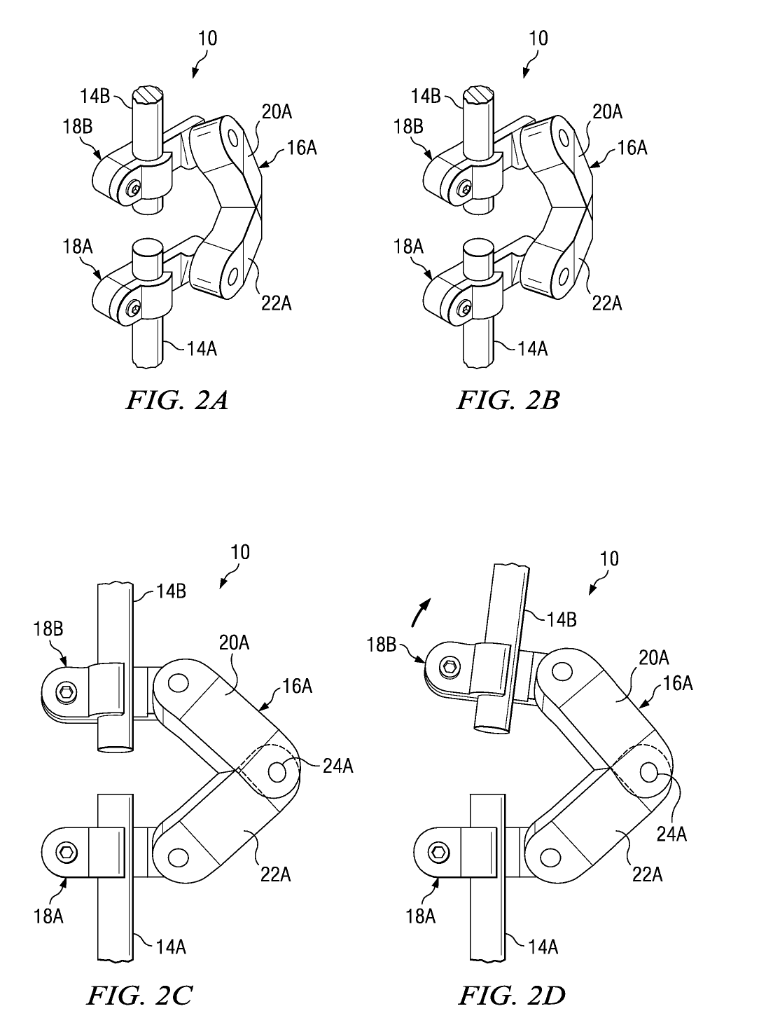

In certain embodiments, one or more dynamic braces 16a-16b may be provided that couple either directly or indirectly with the anchor heads 26a-26c. As illustrated in FIG. 2A, for instance, the brace 16a may couple to the rods 14a and 14b by adjustable connecting members 18a and 18b, respectively. In certain embodiments, the adjustable connecting members 18a-18b enables the brace 16a to be adjusted vertically along the rods 14a-14b to accommodate different distances in pedicle screw placement due to the various anatomies of patients. Furthermore, the adjustable connecting members 18a-18b may rotate about the rods 14a-14b, which in turn may allow the brace 16a to pivot in relation to rods 14a-14b.

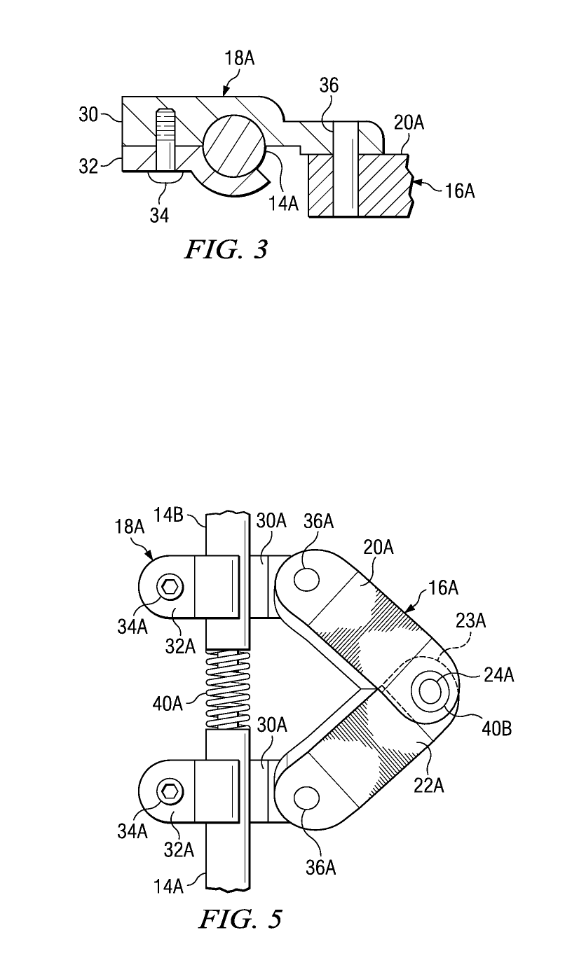

Turning briefly to FIG. 3, there is shown a section view of the connecting member 18a that may be used in certain embodiments. The connecting member 18a may allow the surgeon to adjust the dynamic brace 16a easily once implanted within patient's body. The axial adjustability of connecting member 18a reduces the total number sizes required for the braces 16a (and 16b) in order for surgeons to account for the differences in anatomy among patient populations. The connecting member 18a may comprise a body 30 with an adjustable arm 32 that is sized to receive and clamp the rod 14a. In certain embodiments arm 32 may pivot between an open and closed position to allow for the insertion of rod 14a. The rod 14a may also be able to slide between arm 32 and the body 30 without adjusting the position of arm 32 or the body 30. The arm 32 may be fastened to the body 30 by a fastener 34 such as a screw, bolt, rod, collet or other suitable fastener known to those skilled in the art. When tightened, the fastener 34 may exert a compressive force on the arm 32 which transfers the force to rod 14a rigidly locking rod 14a in place with respect to the connecting member 18a. The connecting member 18a may also allow for an end of a dynamic brace 16a to rotatably couple to the body 30. A fastening device 36 such as a dowel pin, screw, bolt, or other suitable fastening device known to those skilled in the art may be used to secure link member 20a or 22a of the dynamic brace 16A to connecting member 18a while still allowing for rotation of dynamic brace 16a relative to connecting member 18a.

This adjustability will aid in allowing the brace to align or “point” towards a center of rotation, as shown in U.S. patent application Ser. No. 11/443236 entitled “System and Method for Dynamic Stabilization” filed on May 30, 2006, which is hereby incorporated by reference. Alignment tools may also be used to assist with the alignment process. Such tools are described in U.S. Provisional Patent Application 60,711,812, entitled “Dynamic Spinal Stabilization Alignment Instrument,” filed on Aug. 26, 2005; U.S. patent application Ser. No. 11,467,798, entitled “Alignment Instrument for Dynamic Spinal Stabilization Systems,” filed on Aug. 28, 2006; U.S. Provisional Patent Application 60,826,763, entitled “Alignment Instrument for Dynamic Spinal Stabilization Systems,” filed on Sep. 25, 2006, which are herby incorporated by reference for all purposes. Once the desired distance and angle of rotation is achieved the connecting members 18a-18b may be locked in place with respect to the rods 14a-14b by a fastener 34 or other locking means known to those in the art.

In certain embodiments, therefore, the connecting members 18a and 18b may be adjusted so that the bone anchor which is secured to the vertebra may rotate about a center of rotation as more fully described in the PCT Patent Application No. PCT/US2005/027996, entitled, “System and Method for Dynamic Skeletal Stabilization” filed on Aug. 8, 2005. The connecting members 18a-1 8d may also allow the surgeon to adjust or orient the brace 16a-1 6b to prevent the braces 16a-1 6b from interfering with neighboring anatomy of the spine, especially during movement of the spine.

Turning back to FIG. 2A, in the certain embodiments, the dynamic brace 16a may be offset from a longitudinal axis that passes through the adjacent bone anchors. Such an offset may allow for the brace to be larger or more easily placed because the distance and space between adjacent bone anchors and/or vertebrae is limited, especially in smaller patients. In certain embodiments, the dynamic brace 16a may incorporate a first link member 20a and a second link member 22a. In some embodiments, the link members 20a and 22a may be hinged or pivotably connected to each other at their proximal ends, respectively. For instance, the link members 20a and 22a may be secured together by a pin 24a (as shown in FIG. 2C and FIG. 2D). Although a pin 24a is shown, any other suitable fastening device known to those skilled in the art may be used that will allow rotational movement.

Referring to FIGS. 2A through 2D, there is shown one embodiment of the dynamic brace 16a illustrating the range of motion of adjacent vertebrae that may be enabled by the spine stabilization device 10 between two vertebra. FIG. 2A illustrates the brace 16a when the two adjacent vertebrae are in a neutral position. In some situations, the brace 16a may be initially implanted by a surgeon when the patient is in the neutral position. FIG. 2B illustrates the brace 16a when the two adjacent vertebrae move from a neutral to a flexion position (when the patient is bending forward). As the spine moves from a neutral to flexion position the link members 20a and 22a may pivot away from one another, increasing the resulting angle between the two link members. The link members 20a and 22a may also rotate in relation to the rods 14a-14b. FIG. 2C illustrates the brace 16a when the two adjacent vertebrae are in a lateral bending position (when the patient is bending towards the right or left). FIG. 2D illustrates the brace 16a when the two adjacent vertebrae are in a rotational motion (when the patient is turning to the right or left).

As explained above, the relative position and angles of the link members 20a and 22b may be adjusted by moving the connecting members 18a-18b either axially or rotationally and then locking them in place, thus effecting the amount of motion allowed by dynamic brace 16a.

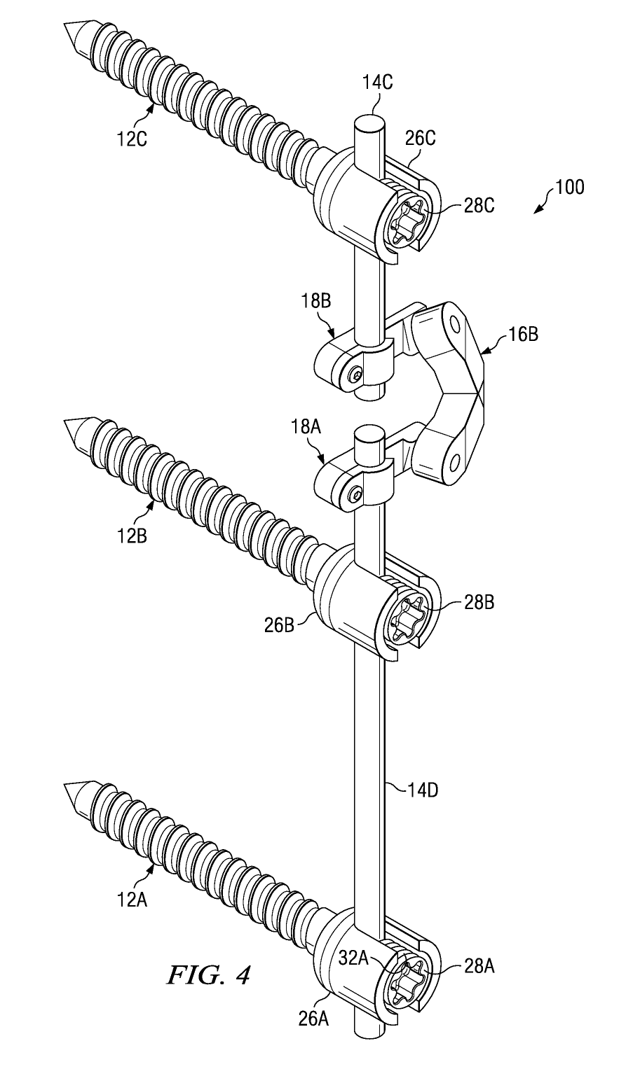

In certain embodiments, one of the dynamic braces 16a or 16b may be replaced by a rigid element such as a rod or a plate that couples to one or more anchor heads (a hybrid system). FIG. 4 illustrates one possible embodiment of a hybrid dynamic stabilization system 100. The hybrid dynamic stabilization system 100 is similar to the dynamic stabilization system 10 as described above, except the lower dynamic brace has been replaced by a rod 14d which may extend from anchor head 26A thru the anchor head 26b to the link member 18a. Thus, this system 100 allows for fusion between the first anchor head 26a and the second anchor head 26b while still allowing for dynamic stabilization between the second anchor head 26b and the third anchor head 26c. Thus, the rod 14d directly links the two anchor heads 26a and 26b. As illustrated, the connecting member 18a may couple the rod 14d to the dynamic brace 16b. Thus, fusion may be achieved at the lower vertebra level with rod 14d, while motion is preserved at the upper vertebra level with the dynamic brace 16b or vice versa. The interchangeability of the multilevel dynamic stabilization system 10 gives a surgeon the desired flexibility required when addressing different clinical needs.

FIG. 5 is a detailed view illustrating one component which may be incorporated in dynamic stabilization system 10 to limit the amount of movement allowed by the dynamic brace 16a. In order to stabilize adjacent vertebrae, a force must be applied to the vertebra to keep them separated during movement of the spine. The force may increase or decrease as the spine moves through its natural motions. In certain embodiments, a limiter element 40a (or 40b) may act to apply a force to aid in the distraction of adjacent vertebrae by limiting or applying a force on the dynamic brace 16a-16b in either extension or flexion, or both. The limiter element 40a (or 40b) may incorporate additional elements to aid in either flexion or extension. For example, FIG. 5 shows the limiter element 40a which may work in conjunction with the limiter element 40b. In this illustrative example, the limiter element 40a may be a helical spring or isomeric dampener positioned between the ends of the rods 14b and 14b. The limiter element 40b may be a torsional spring coupled to the joint 23a. In other embodiments, the limiter elements 40a (or 40b) may be a spring (such as a torsion spring, leaf spring or compression spring), a tension band, bumper or other device that limits or controls the force acting on the dynamic brace 16a-16b either in flexion and/or extension of the spine. In certain embodiments, one limiter element 40a (or 40b) may apply a force during flexion of the spine while the other limiter 40a (or 40b) applies a force during extension of the spine. Both limiters 40a-40b may also apply a limiting force during rotation and lateral bending of the spine.

In certain embodiments, the limiter element 40a or 40b may incorporate a soft or a hard stop. For example the complete compression of a spring (or a spring with a certain spring constant) may provide a stop that prevents any further movement of the spine in either extension or flexion (or rotation or lateral bend). The limiter elements 40a-40b may also be so rigid as to allow very little or no motion of dynamic braces 16a-16b which may aid in promoting fusion of the attached vertebrae. A locking element may also be provided, such as a set screw, to convert the dynamic braces 16a-16b to a fusion brace by restricting any motion.

In yet other embodiments, for instance, the pin element 24a may be replaced with a locking element which effectively converts the dynamic brace to a rigid element might be provided. Thus, at a later date, the surgeon may quickly convert the dynamic brace into a static or fused brace.

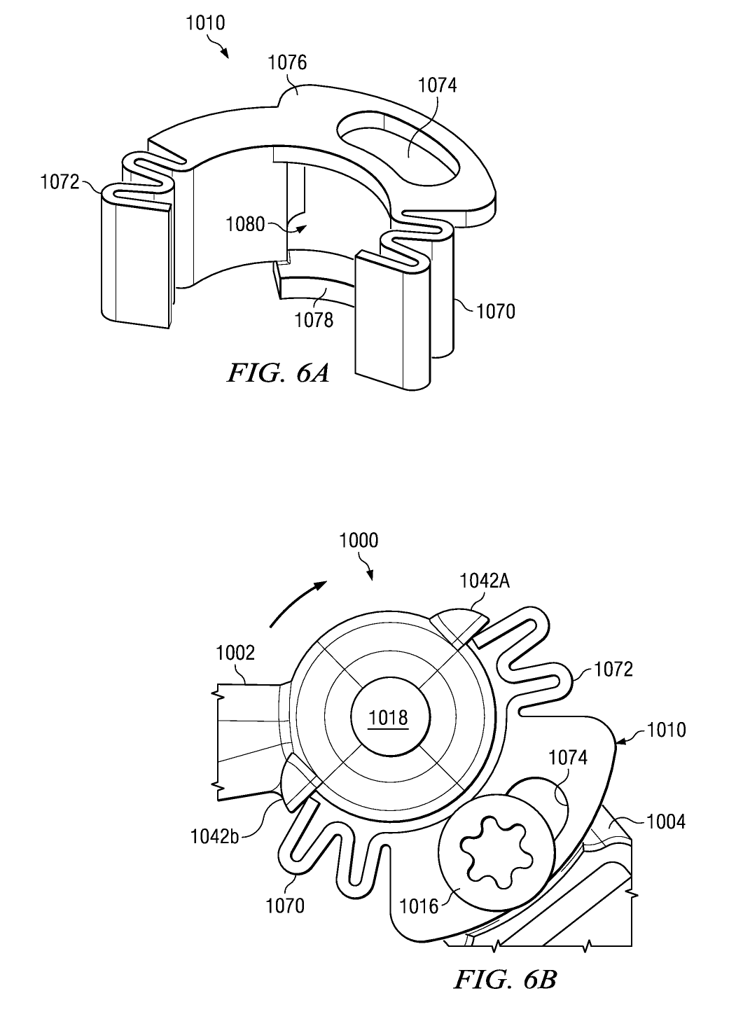

FIG. 6A and FIG. 6B illustrate an alternate embodiment of a dynamic brace and a limiter or torsion spring, as described more fully in U.S. Provisional Application 60/883,314 entitled “Dynamic Linking Member for Spine Stabilization System” filed on Jan. 3, 2007, the disclosure of which is hereby incorporated by reference. A limiter or torsion spring 1010 may be incorporated into a dynamic brace 1000 to control the force required exerted between a first linking member 1002 and a second linking member 1004 of the dynamic brace. The first linking member 1002 and the second linking member 1004 may be pivotably coupled to each other with pin 1018. In this embodiment, the limiter 1010 may have a top wall 1076 and a bottom wall 1078 with an open space 1080 in-between. The top 1076 and bottom 1078 walls may be connected by two opposite side walls which have dampening members 1070 and 1072 that extend along the longitudinal axis of the limiter 1010. In the present example, the dampening members 1070 and 1072 extend along a curved or arcuate longitudinal axis. The space 1080 in-between the top 1076 and bottom 1078 walls of torsion spring 1010 may be dimensioned to receive a shaped end of one of the linking members of the dynamic brace.

FIG. 6B shows an enlarged front view of one embodiment of the joint of linking members 1002 and 1004 assembled with the limiter 1010. In certain embodiments, the limiter 1010 may have a slot 1074 that extends through its top wall 1076. Slot 1074 of the limiter 1010 may align with a hole on the second linking member 1004. The tension of the limiter 1010 may be adjusted by adjusting the position of the slot 1074 relative to the hole on the second linking member 1004 and the limiter adjustment member 1016. The limiter adjustment member 1016 may be inserted through slot and into the hole of second linking member 1004 to secure the limiter to the second linking member 1004.

The distal end of the dampening members 1070 and 1072 may mate or contact protrusions 1042a and 1042b of first linking member 1002. Dampening members 1070 and 1072 may exert a force against protrusions 1042a and 1042b, respectively. As the first and second linking members 1002 and 1004 move towards each other (as shown by large arrow in FIG. 6B), one dampening member 1070 may compress against protrusion 1042b, while other dampening member 1072 may relax or extend, as shown in FIG. 6B. Dampening member 1072 may compress and exert a force against protrusion 1042a, if first and second linking members 1002 and 1004 are moved in the opposite direction. The amount of force exerted on protrusions 1042a and 1042b by dampening members 1070 and 1072 (respectively) may be adjusted by adjusting the position of slot 1074 relative to limiter adjustment member 1016. For example, if member 1016 is positioned further away from one end of slot 1074, as shown in FIG. 6B then dampening member 1072 may be compressed more (and member 1074 may be compressed less) than if member 1016 was positioned in the middle (or at the other end) of slot 1074.

In certain embodiments the limiter spring 1010 may be molded or machined from an elastomeric or polymeric material. Dampening members 1070 and 1072 may be molded or machined from the same material as the rest of torsions spring 1010 or may be manufactured from a metallic material such as nitinol, stainless steel or titanium. Dampening members 1070 and 1072 may achieve its dampening characteristics through its wave-like design as shown in FIG. 6A and/or the material properties of the material it is manufactured from. In other embodiments, dampening members 1070 and 1072 may include various types of springs designs, such as torsion springs, compression springs or wave springs.

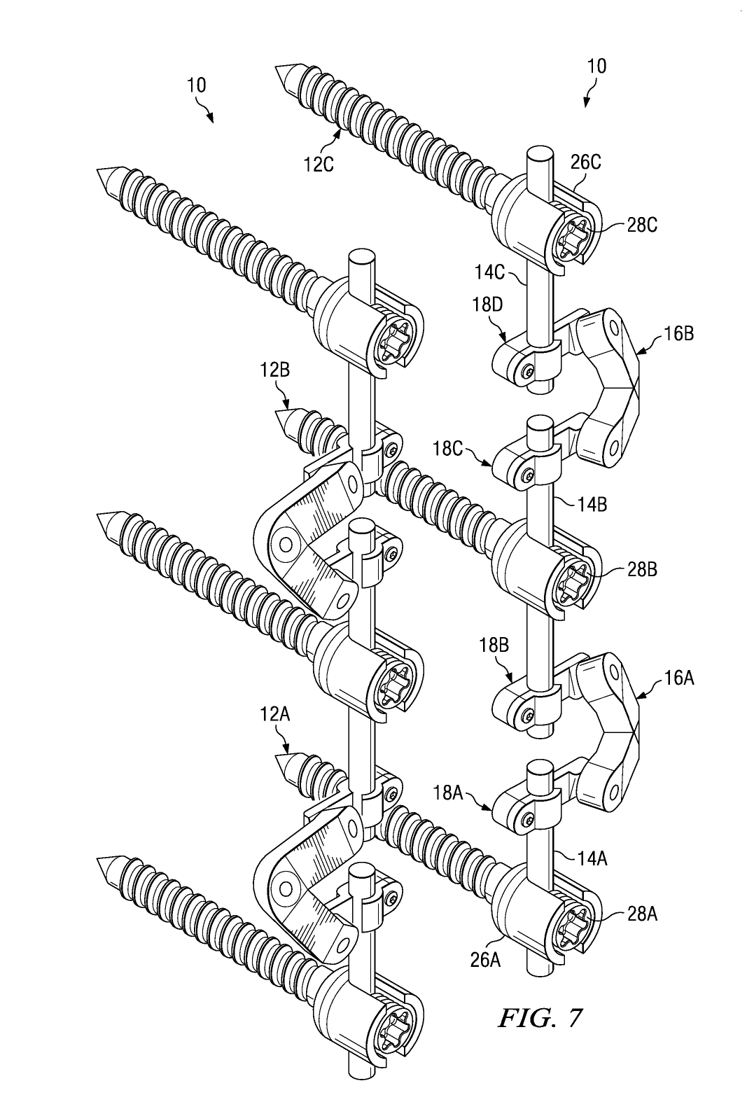

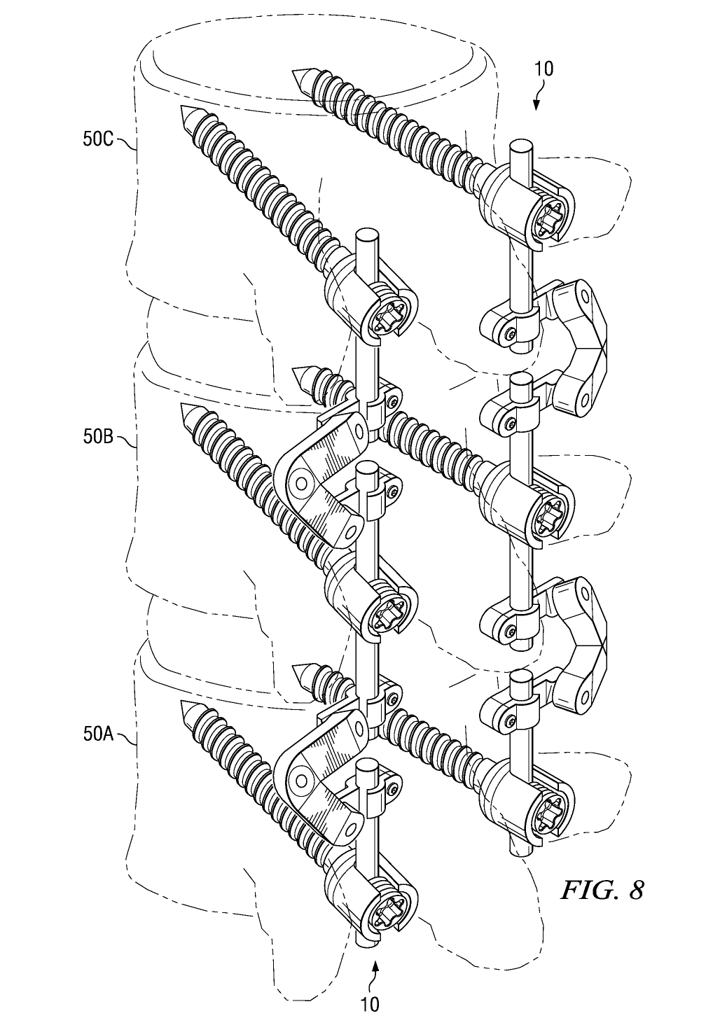

It is understood that the various components described above such as bone anchor 12a-12c, anchor heads 26a-26c, rod 14a-14c, braces 16a-16b, connecting member 18a-18d, dampening element 40a and 40b and locking member 28a-c may be assembled together as required by a surgeon to create a dynamic stabilization system 10. These components are interchangeable and some components may not be used in a system and some components may be used more than once. FIG. 7 illustrates one example of a multi-level dynamic stabilization system 10 for securing to both sides of the spinous process. As illustrated, both sides of the spine stabilization system are similar, but as described above, depending on the patient needs, a surgeon may construct a different multi-level dynamic stabilization system on either side of the spine as illustrated in FIG. 8.

In the example illustrated in FIG. 7 a bone anchor 12a may be multiaxially coupled to a anchor head 26a, which may receive a rod 14a that may be coupled to connecting member 18a. Connecting member 18a may be coupled to one side of dynamic brace 16a. A second connecting member 18b may be used to couple to an opposing side of dynamic brace 16a. Second connecting member 18b may be coupled to a second rod 14b which may slide into a slot of a second anchor head 26b which is coupled to a second bone anchor 12b which is secured within a vertebra of the next level. Second rod 14b may extend through the slot in second anchor head 26b. The end portion of second rod 14b may couple to a third connecting member 18c which couples to a side of a second brace 16b. The opposing side of second brace 16b may couple to a fourth connecting member 18d which may receive a third rod 14c. The third rod 14c may then couple to a third anchor head 26c which is multi-axially coupled to a third bone anchor 12c which is secured to the next level vertebra.

The system may be implanted in either an open or a minimally invasive manner. Furthermore, either the entire system or portion of the system may be assembled outside the body and adjusted once implanted. The surgeon may slide rods 14a-14c within anchor heads 26a-26c and may slide and/or rotate the connecting members 18a-18d along the rods 14a-14c until the desired orientation of the dynamic braces 16a-16b is achieved (for example the dynamic brace(s) points toward the center of rotation for that vertebral level). The surgeon may also adjust the anchor heads 26a-26c to achieve the desired orientation of the dynamic stabilization system 10. Once the desired position is achieved the locking members 28a-28c and connecting members 18a-18d may be tightened to fix the orientation of the dynamic stabilization system 10. This process of connecting various components of the dynamic spinal stabilization system 10 may be continued along the spine to additional levels (including cervical vertebrae). A second dynamic stabilization system 10 may be implanted on the opposite side of the spine for multiple levels (three or more vertebrae) as is shown in FIG. 7. FIG. 8 further illustrates both sides of the spine stabilization device as implanted into multiple vertebrae 50a-50c. The dynamic stabilization system as shown in FIG. 7 and FIG. 8 may be easily modified. For example, the limiter element 40a-40b may be added to any number of dynamic braces 16a (or 16b) as described above.

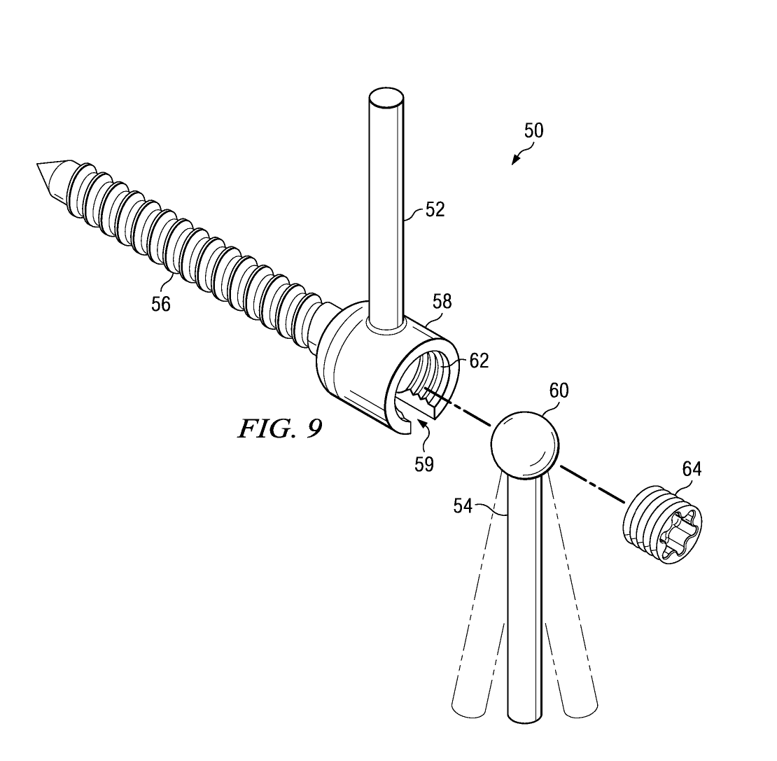

FIG. 9 illustrates an alternative embodiment 50 showing another coupling mechanism. Rods 14a-14c described above may be substituted for rods 52 and 54. In this alternative embodiment, the rod 52 may be fixedly secured to the screw head 58. In certain embodiments, there may be a side slot 59 formed within a wall of the screw head 58. As illustrated, the rod 54 may have an enlarged or spherical portion 60. Spherical portion 60 may be received by and fit within a proximal opening 62 of the screw head 58. Screw head 58 may be multiaxially coupled to bone anchor 56 as described earlier. The combination of spherical portion 60 and multiaxial screw head 58 allows for two points of adjustability at the same location. In other words, when a single rod is used (e.g. rod 14b as illustrated in FIG. 1), the position and alignment of the rod must be “balanced” between the adjacent levels. However, if a single rod is replaced with the screw head 58 and a rod 54, each of the respective ends may be adjusted independently. This flexibility allows components to line up easier and in the desired orientation.

In certain embodiments, the spherical portion 60 allows the rod 54 to be rotated and angularly positioned with respect to the screw head 58. Screw head 58 may then also be rotated and angularly position independent of the rod 54 and the bone anchor 56. Once properly positioned, the rod 54 may be secured to the screw head 58 with a locking means, such as a cap screw 64. Although rods are described in the various embodiments, these rods may also include plates as well as rods of various cross sectional geometries.

In certain embodiments the rod 54 may extend along a horizontal axis that is substantially perpendicular to a longitudinal axis of the bone anchor 56. In other embodiments the rod 54 may extend at an angle to the horizontal axis. For example the rod may extend at an angle of 40 degrees below the horizontal axis to an angle of 40 degrees above the horizontal axis.

Thus, in the alternative embodiment 50, there is an additional degree of freedom which allows the rods to be individually angularly positioned with respect to each other. Such an additional degree of freedom may allow the dynamic brace or fusion rods for each level to be more easily adjusted so that each brace may be aligned with its respective center of rotation.

In other embodiments screw head 58 may have a U shaped channel and rods 52 and 54 may be one component with a spherical portion located between its ends. In such an embodiment the spherical portion may include a single ball or two partial spheres that are located within screw head 58. As stated above screw head 58 may also be multiaxially coupled to a bone anchor.

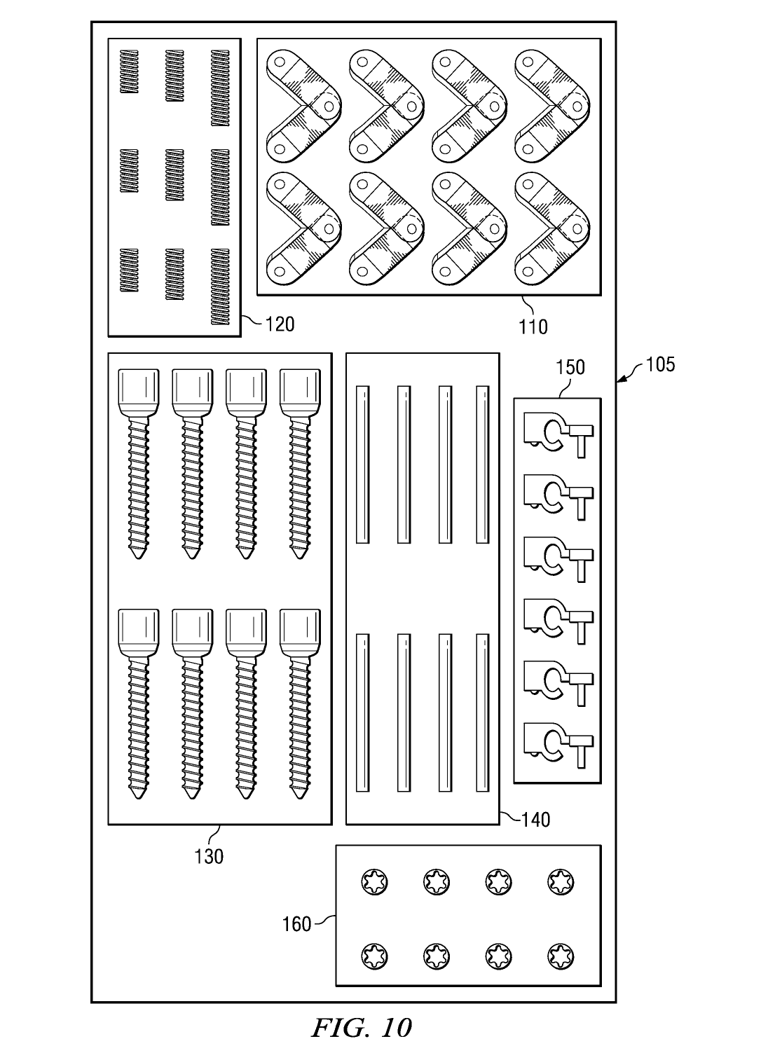

Referring now to FIG. 10 one embodiment of a multi-level dynamic stabilization kit 105 is shown. The kit 105 may include a plurality of any component described above, including a plurality of dynamic braces 110, a plurality of rods of 140, a plurality of connecting members 150, a plurality of bone anchors with multi-axial heads pre attached 130, a plurality of locking members 160, and a plurality of limiter elements 120. The limiter elements 120 in the kit 105 may have varying compressive and/or extension forces as to allow the surgeon to choose the amount force needed for the vertebrae to move in extension and flexion (as well as rotation and lateral bend). The rods 140 in the kit may consist of several rods having various lengths and/or bends which enables the surgeon to customize the multi-level dynamic stabilization system implanted. The kit 105 may also incorporate a tray with inserts for the various components. In certain embodiments the tray may be sterilizable and manufactured from metal or high temperature plastic. In other embodiments the surgeon may order a pre-sterile pack with all of the required implants in the pack to assemble the desired dynamic multi-level system.

The foregoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

For instance, in some embodiments, there may be a spine stabilization device comprising a plurality of rods coupled to bone anchors wherein each bone anchor is secured to one rod in a polyaxial manner. The spine stabilization device may further comprise braces rotatably coupled between rods by a connecting member, the braces comprising two spherical link members coupled together at the proximal ends thereof by a fastener such as a pin or a screw.

In other embodiments, there may be a spine stabilization device wherein the braces are coupled together at the proximal ends thereof by a hinged mechanism.

In yet other embodiments, there may be a spine stabilization device may further comprise a spring coupled between the rods for added flexibility and stability.

In other embodiments, there may be spine stabilization device comprising: a plurality of rods; a plurality of bone anchors, wherein each bone anchor may be secured to one rod in a polyaxial manner; at least one end of each rod rotatably coupled to a brace adapted to span between two bone anchors; each brace comprising a first and second link member wherein the distal end of the first link member rotatably secures to a first rod near the bottom end thereof, the distal end of the second link member rotatably secures to a second rod near the upper end thereof, and the first and second link members are pivotably secured to each other at the proximal end thereof; means for securing the first and second link members together; and means for securing the brace to the rods; wherein the brace allows for movement between the first link member and the second link member such that the movement of the second link member with respect to the first link member is generally restricted to a three dimensional curved path having a substantially constant radius about a center of rotation positioned outside of the brace.

Additionally, the means for securing the first and second link members together comprises a pin and the means for securing the brace to the rods is first and second link members together comprises a connecting member. In some embodiments, the connecting member comprises a body, an adjustable arm, means for securing the arm to the body, and means for securing the link member of the brace to the body of the connecting member.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods or steps, presently existing or later to be developed that perform substantially the same function or achieve substantially the same result as the corresponding embodiments described herein may be utilized. Accordingly the appended claims are intended to include within their scope such processes machines, manufacture, compositions of matter, means, methods or steps.

What is claimed is:

1. A dynamic multi-level spine stabilization system comprising:
a bone anchor comprising a distal vertebral anchoring portion and a proximal head portion;
a first anchor head multi-axially coupled to proximal head portion of the bone anchor, the anchor head having an external surface and a threaded internal surface, and a thru hole extending along the central axis, the anchor head having a C shaped cross section created by a longitudinal slot extending into the anchor head in a direction generally perpendicular to the through hole, the anchor head having an integral elongated member extending in a direction substantially perpendicular to the central axis;
a first dynamic brace coupled to the integral elongated member;
an adapter having a spherical shaped proximal portion coupled within the anchor head and a distal portion;
a second dynamic brace coupled to the distal portion of the adapter; and
a locking cap comprising a threaded external surface coupled to the threaded internal surface of the anchor head and a bottom surface rigidly coupled to the spherical shaped proximal portion.
